(19) United States
(12) Patent Application Publication
(10) Pub. No.: US 2005/0154245 A1
(43) Pub. Date: Jul. 14, 2005

(54) Hydrogen recovery in a distributed distillation system

(76) Inventors: Rian Reyneke, Katy, TX (US); Iain Sinclair, Walton (GB); Guang-Chung Lee, Houston, TX (US); Michael J. Floral, Aurora, IL (US); Jeffery S. Logsdon, Naperville, IL (US); Wayne W. Y. Eng, League City, TX (US); Christos G. Papadopoulos, Naperville, IL (US)

Correspondence Address: BP America Inc.;Docket Clerk, BP Legal, M.C. 5-East, 4101 Winfield Road, Warrenville, IL, 60555, US

(21) Appl. No.: 10/740,273

(22) Filed: Dec. 18, 2003

(51) Int. Cl. C07C 7/04

(52) U.S. Cl. 585/800

(57) ABSTRACT

A process for recovering hydrogen from a mixed hydrocarbon stream wherein the mixed hydrocarbon stream is subjected to a separation technique to produce a substantially hydrogen enriched stream, which is then recovered as hydrogen product. A process for providing refrigeration duty to the process is also disclosed, wherein a substantially methane enriched stream arising from the separation technique is expanded to provide cooling duty for the process.

### STATEMENT REGARDING FEDERALLY SPONSORED RESEARCH OR DEVELOPMENT

This invention was made with government support under United States Department of Energy Cooperative Agreement No. DE-FC07-01ID14090.

### CROSS REFERENCE TO RELATED APPLICATIONS

None.

### BACKGROUND OF THE INVENTION

For many years, distributed distillation has been suggested as a basis for the design of refinery systems, ethylene recovery systems, and other commercial chemical, petroleum, and petrochemical separations systems. Distributed distillation is best understood by contrasting it with sharp split distillation. In sharp split distillation, a separation is made between light and heavy components that are adjacent to each other on the volatility curve of the mixture being separated. That is, there are little or no compounds in the mixture that have a volatility that is intermediate to those of the light and heavy components.

For example, a typical sharp split deethanizer column in an ethylene recovery system performs a sharp split between ethane and propylene. The overheads of the column contain essentially no propylene and the bottoms contain essentially no ethane. The overheads therefore contain all components lighter than the light component (e.g. ethylene, methane, etc.), and the bottoms contain all components heavier than the heavy component (e.g. propane, C4+, etc).

In a distributed distillation operation, a sharp split is not made between components that are adjacent on the volatility curve. A distributed distillation analog to the deethanizer is a “C2 distributor”. A C2 distributor column produces a sharp split between methane and C3 components while distributing ethane and ethylene between the column overhead and bottoms. In a C2 distributor column, the light component is methane and the heavy component is propylene. These components are not adjacent to each other on the volatility curve; ethane and ethylene have a volatility that is intermediate between methane and propylene. In this case, then, ethane and ethylene distribute between the column overheads and bottoms. The overheads contain some ethane and ethylene, as well as methane and lighter components, but essentially no propylene. The bottoms also contain some ethane and ethylene, as well as propylene and heavier components, but essentially no methane. Of course, further purification of the components is done in downstream columns.

A benefit of a distributed distillation system is that it requires less total energy to produce the final purified components than an analogous sharp split distillation sequence. A way of understanding the energy savings provided by distributed distillation is that it accomplishes the separation of components with fewer overall phase changes. Phase changes (condensation or vaporization) require energy, and reducing the number of phase changes also reduces the energy consumption of the system.

U.S. Pat. No. 5,675,054 issued to Manley, and Manley and Hahesy (Hydrocarbon Processing, April 1999 p. 117) describe distributed distillation in ethylene recovery and purification. Manley describes the use of an ethylene distributor column in which both the bottoms and overheads stream contain ethylene, and in which a product-quality separation of ethane and ethylene in the overhead product is achieved.

In Manley '054, the ethylene distributor is thermally coupled to the downstream demethanizer column. The vapors from the ethylene distributor flow directly into the demethanizer for further separation, while a liquid side draw from the demethanizer is used to reflux the ethylene distributor column. The overheads of the demethanizer column contain primarily hydrogen and methane, and can be further treated to recover methane and hydrogen if desired. The Hydrocarbon Processing article states that after removing sufficient ethylene from the demethanizer overhead stream, it can be sent to a standard adiabatic hydrogen purification section where Joule-Thompson expansion is utilized to further chill and separate a purified hydrogen product from the demethanizer overhead stream.

U.S. Pat. No. 5,035,732 issued to McHue is directed to a process for recovering C2 hydrocarbons from a mixed stream that utilizes dephlegmation of the feed and introduction of the dephlegmator liquid product into a demethanization system, characterized by the presence of two demethanization steps, one at moderately low cryogenic temperatures and one at very low cryogenic temperatures.

U.S. Pat. No. 4,629,484 issued to Kister is directed to cooling a gaseous hydrocarbon feed mixture in a plurality of cooling stages and introducing a bottoms portion from at least one cooling stage to a hydrogen stripper, in which hydrogen is removed from the bottoms portion before the bottoms portion is introduced to a demethanizer fractionating column, and where relatively pure hydrogen and methane streams are produced.

U.S. Pat. No. 4,900,347 issued to McHue is directed to a process for recovering ethylene from cracked hydrocarbon feed gas which utilizes a plurality of dephlegmator units, the liquid products of which are fed into serially connected demethanizer fractionators.

Surprisingly, we have found that making a rough separation of methane and hydrogen downstream of the ethylene distributor and upstream of the hydrogen recovery and purification section of the plant significantly increases the hydrogen recovery of the process with only a small increase in energy levels. In contrast to standard distributed distillation systems, a hydrogen depleted gas is expanded and used for refrigeration, so less hydrogen is degraded from chemical to fuel value. This overcomes one of the disadvantages of a typical distributed distillation system, namely, low hydrogen recovery. We have further found that the methane rich gas from the aforesaid rough separation can be expanded and chilled to provide a cooling duty to the overall process.

### SUMMARY OF THE INVENTION

This invention describes a process for recovering hydrogen. The process comprises the steps of introducing a mixed hydrocarbon feed stream comprising a mixture of hydrogen, methane, and C2 components into an ethylene distributor column to obtain a first stream. This first stream is subjected to a separation technique to produce a second stream substantially hydrogen enriched and substantially methane depleted, and a third stream substantially hydrogen depleted and substantially methane enriched. A purified hydrogen product is recovered from the second, substantially hydrogen enriched and substantially methane depleted stream.

This invention also describes a process for recovering hydrogen and recovering refrigeration duty. The process comprises the steps of introducing a mixed hydrocarbon feed stream comprising a mixture of hydrogen, methane, and C2 components into an ethylene distributor to obtain a first stream. The first stream is subjected to a separation technique to produce a second stream substantially hydrogen enriched and substantially methane depleted, and a third stream substantially hydrogen depleted and substantially methane enriched. This third stream is introduced into a second separation column to recover a fourth stream substantially methane enriched. This fourth stream is directed to an expansion device to produce a lower-temperature, lower pressure fifth stream. This fifth stream is reheated to provide cooling duty to the overall process. A purified hydrogen product is recovered from said second stream.

The process shall be described for the purposes of illustration only in connection with certain embodiments. However, it is recognized that various changes, additions, improvements, and modifications to the illustrated embodiments may be made by those persons skilled in the art, all falling within the scope and spirit of the invention.

### BRIEF DESCRIPTION OF DRAWINGS

FIG. 1 depicts an olefins plant demethanization section that utilizes an ethylene distributor and demethanizer tower to depict the hydrogen recovery of this invention.

FIG. 2 depicts an embodiment which utilizes dephelgmator technology to provide the partial separation of hydrogen and methane between the ethylene distributor overhead and the demethanizer inlet.

### DETAILED DESCRIPTION OF THE INVENTION

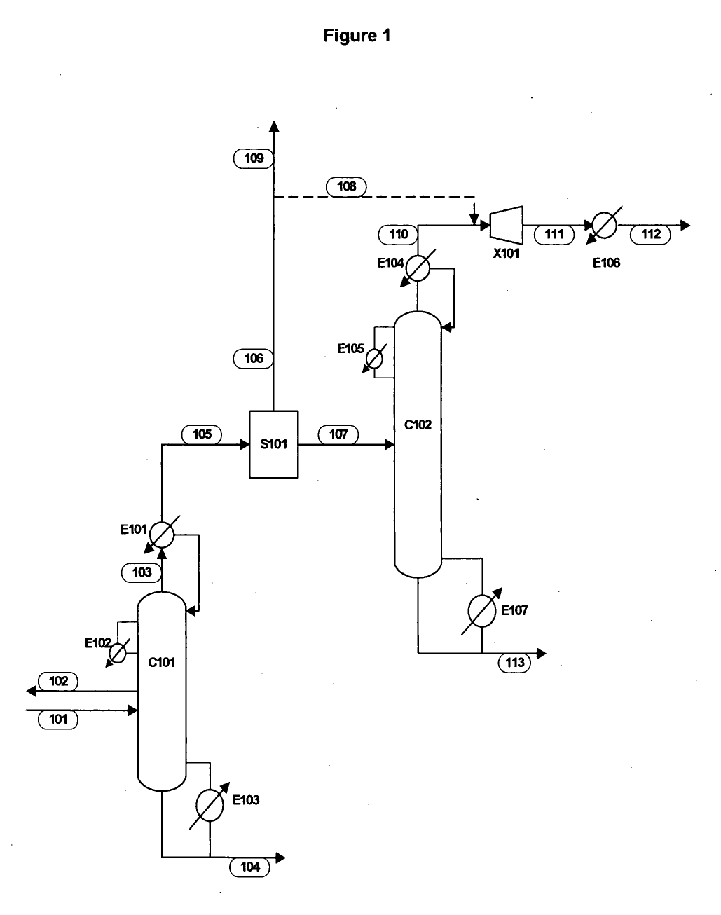

With reference to FIG. 1, the feed to the process enters as stream 101. Stream 101 comprises a mixture of hydrogen, methane, ethane, and ethylene and optionally heavier hydrocarbons. Stream 101 enters C101, which serves as an ethylene distributor. A liquid side stream, stream 102, can optionally be withdrawn from C101 at or near the feed point to provide reflux to an upstream column or columns. The overhead stream arising from C101, stream 103, comprises essentially all of the hydrogen and methane that enters the column as well as a fraction of the ethylene. Stream 103 enters a partial condenser, E101, which provides at least a portion of the reflux to column C101. One or more side condensers can optionally be utilized on C101, such as E102 as shown. Column C101 is controlled such that the ethane/ethylene ratio in stream 103 is sufficiently low that a product quality ethylene can be produced from this stream by removal of the hydrogen and methane. Typically, the ethane content of stream 103 is controlled by adjusting the reflux liquid provided to C101 by exchangers E101 and/or E102.

The bottoms product of C101, stream 104, comprises ethane and heavier hydrocarbons leaving column C101, as well as the remaining fraction of the ethylene. Stream 104 can be further purified in downstream columns. FIG. 1 shows column C101 operated with a conventional reboiler exchanger, E103. Column C101 is typically operated so that there is essentially little or no methane in stream 104. The methane content of stream 104 can be controlled by adjusting the amount of stripping vapor provided to column C101 by exchanger E103.

In FIG. 1, the uncondensed vapor from E101, shown as stream 105, is sent to a hydrogen/methane separation step, designated S101 in FIG. 1. In this step, at least a partial separation is made between the hydrogen and the heavier components (primarily methane and ethylene) present in the net overhead vapor from C101. This separation step can include the use of partial condensation and separation, rectification, membrane separation, dephlegmation, and adsorption, individually or in a combination of these or other operations known to those in the art to be beneficial for separation of hydrogen from a mixed hydrocarbon stream.

The separation step S101 produces two streams. The first stream, stream 106, is enriched in hydrogen relative to stream 105. The second stream, stream 107, is depleted in hydrogen relative to stream 105. A portion of stream 106 can be optionally fed to an expander system as shown by dotted line 108. The remainder of the hydrogen-enriched stream, stream 109, would advantageously be sold as product hydrogen or sent to a further hydrogen purification step if needed. Beneficially, the flow in stream 108 would be minimized in order to maximize the fraction of the hydrogen-enriched stream 106, which is sent to the hydrogen product recovery via stream 109. In some cases, however, the refrigeration requirement for the overall process and the process energy balance will dictate that some of stream 106 be directed to the expander system, X101, via stream 108. The material from stream 108 would provide additional chilled expander outlet gas which would in turn to provide the required refrigeration duty as described below.

Stream 107 is fed to column C102, which serves as a demethanizer. If C102 operates at a pressure significantly lower than that of S101, a valve or other expansion device may be employed in line 107. The overheads of C102 are partially condensed in E104 to provide reflux to the tower C102. FIG. 1 shows this to be accomplished by a standard partial condenser arrangement, though other means, such as dephlegmation, could also be used. One or more side condensers can be utilized on C102, shown as exchanger E105, to provide additional reflux liquid to the tower. The uncondensed vapor from E104, stream 110, is the net overhead product from the demethanizer and comprises methane, optionally hydrogen, and very little ethylene. Stream 110 is fed to an expander system X101 for recovery of refrigeration. FIG. 1 shows a single stage of work expansion being accomplished on the net demethanizer overhead to provide a chilled lower-pressure stream, stream 111. This chilled lower-pressure stream 111 is reheated against other process or external refrigeration streams to provide cooling duty to the rest of the process. FIG. 1 shows this being done in a single exchanger E106, though multiple exchangers can also be used. A Joule-Thompson expansion could also be employed in place of the work expansion device that is shown, as is well known to those skilled in the art. The final reheated expanded stream, stream 112, comprises primarily methane and some hydrogen and can be used as plant fuel if desired. The bottoms product of C102, stream 113, comprises product-quality ethylene.

FIG. 1 shows column C102 being operated with a conventional reboiler exchanger E107. Column C102 is typically operated so that there is essentially little or no methane in stream 113. The methane content of stream 113 can be controlled by adjusting the amount of stripping vapor provided to column C102 from exchanger E107. A benefit presented by this invention is that of a partial separation of hydrogen and methane that takes place between the ethylene distributor and demethanizer. This results in production of a stream, stream 110, that is relatively depleted in hydrogen, which can be preferentially fed to the expander system to provide refrigeration for the rest of the process. As a result, the amount of the hydrogen-enriched stream, stream 108, that is directed to the expanders is reduced or eliminated, and hydrogen loss to fuel is therefore reduced. An additional benefit of this invention is the lack of thermal coupling between the ethylene distributor and the demethanizer columns. The elimination of thermal coupling between these two columns allows the columns to operate more independently, each with its own condenser for providing reflux. This improves the controllability of the system compared to the prior art by providing more direct control of the column overhead compositions. Another benefit from this arrangement is that the demethanizer overheads are depleted in hydrogen relative to the prior art and thus the temperature of the top of the demethanizer is higher than in the prior art. This means that relatively cheaper, higher-temperature refrigeration can be used for the demethanizer condenser compared with the prior art.

The process shown in FIG. 1 has significant benefits over the prior art in terms of hydrogen recovery. This invention recovers significantly more hydrogen than the prior art, and those skilled in the art will recognize that the reason for the improved hydrogen recovery is the partial separation of hydrogen and methane that occurs between the ethylene distributor and the demethanizer towers, which allows preferential feeding of a relatively hydrogen-depleted stream to the expansion system for recovery of refrigeration.

### EXAMPLE

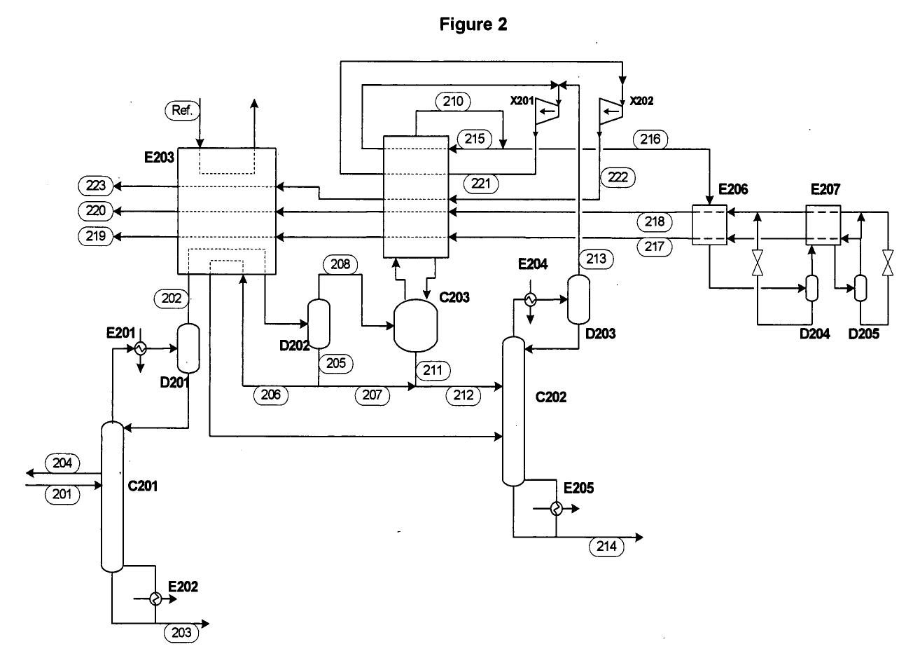

FIG. 2 depicts an embodiment which utilizes dephelgmator technology to provide the partial separation of hydrogen and methane between the ethylene distributor overhead and the demethanizer inlet.

A mixed hydrocarbon feed, stream 201, enters column C201. In FIG. 2, stream 201 is the chilled overhead vapor from a deethanizer column (not shown) and comprises a mixture of hydrogen, methane, ethylene, and ethane and optionally heavier hydrocarbons. Column 201 contains 105 theoretical trays, and the feed, stream 201, enters on theoretical tray 75 (as measured from the top). The overhead vapor of column C201 is partially condensed in exchanger E201. The liquid and vapor from exchanger E201 are separated in drum D201 and the liquid is returned to C201 as reflux. Stripping vapor is provided to the bottom of C201 by reboiler E202.

Column C201 serves as an ethylene distributor, making a sharp split between ethane and methane, and allowing ethylene to distribute between the overhead and bottom streams. The overhead vapor from D201, stream 202, comprises a mixture of hydrogen, methane and ethylene, but little, if any, ethane. The bottoms liquid, stream 203, comprises a mixture of ethane and ethylene, but little, if any, hydrogen or methane. Stream 203 can be further purified in downstream columns. A liquid sidestream, stream 204, is taken from C201 at a point just above the feed. Stream 204 is used as reflux liquid to the upstream deethanizer tower (not shown).

Stream 202 is further chilled to about −145° F. in exchanger E203 against an external refrigerant circuit, shown as stream Ref, and reheating hydrocarbon vapors as described below. The partially condensed stream is sent to separator drum D202. The liquid from D202, stream 205, is split into two streams. One portion, stream 206, is reheated to about −45° F. in E203 and then sent to the demethanizer column C202. The remaining portion, stream 207, is sent directly to the demethanizer column C202.

The vapor from D202, stream 208, is sent to dephlegmator C203. The dephlegmator C203 is chilled with a variety of reheat streams as described below. Within the dephlegmator C203, both heat and mass transfer operations are carried out. The dephlegmator C203 has been simulated as having ten theoretical separation stages, with equal heat removal at each stage. The overhead vapor from C203, stream 210, is enriched in hydrogen and depleted in methane and ethylene. Stream 210 is sent to further purification and recovery of a salable hydrogen product as described below. The bottoms liquid from C203, stream 211, is combined with stream 207 to become stream 212 and sent to the demethanizer column C202.

The demethanizer column C202 contains 45 theoretical stages. The upper feed, stream 212, enters at theoretical stage 9 (as measured from the top), and the lower feed, reheated stream 206, enters at theoretical stage 15. The overhead vapor of column C202 is partially condensed in exchanger E204. The liquid and vapor from E204 are separated in drum D203 and the liquid is returned to C202 as reflux. Stripping vapor is provided to the bottom of C202 by reboiler E205. The overhead vapor from D203, stream 213, is enriched in methane and contains little, if any, ethylene. Stream 213 is sent to an expansion device to provide cooling for the dephlegmator C203 and other process cooling as described below. The bottoms liquid from C202, stream 214, contains product-purity ethylene.

The hydrogen-enriched stream emerging from the overhead of the dephlegmator C203, stream 210, is split. In this example the process cooling requirements are greater than can be supplied by expansion of only the demethanizer overhead vapor which is described below. Therefore, a minor fraction of stream 210, designated stream 215, is reheated in the dephlegmator C203 and then sent to the first stage expander X201 to provide additional chilled expander outlet vapor and therefore additional process cooling capacity. The majority of stream 210, designated stream 216, is sent to hydrogen recovery. A typical 2-stage adiabatic hydrogen recovery section is shown in FIG. 2. It consists of two heat exchangers, E206 and E207, and two drums, D204 and D205. The operation of the hydrogen recovery section is well known to those skilled in the art and will not be described in detail here. The operation results in a high-pressure, high-purity hydrogen stream, stream 217, and a lower-pressure methane-rich fuel gas stream, stream 218. These streams are reheated first through the dephlegmator C203 and then through E203 as shown. These streams will typically also be further reheated elsewhere in the process to recover additional cooling capacity. The reheated hydrogen product stream is designated stream 219 and the reheated lower-pressure fuel stream is designated stream 220.

The overhead vapor from the demethanizer column, stream 213, is directed, along with a minor fraction of the hydrogen-enriched stream, stream 215, to the first expansion stage X201. The expander reduces the pressure of the stream to an intermediate pressure, thereby cooling the stream. The cold intermediate-pressure stream, stream 221, is reheated through the dephlegmator C203, and then sent to the second stage expander, X202. This second expander reduces the pressure of reheated stream 221 to a lower pressure, further cooling it. The cold lower-pressure stream, stream 222, is also reheated through the dephlegmator C203, and then through exchanger E203. This stream will typically be further reheated elsewhere in the process to recover additional cooling capacity and then used as fuel. The reheated second lower-pressure fuel stream is designated as stream 223. Those skilled in the art will recognize that the expanders X201 and X202 could be part of expander/compressor (compander) sets.

Table 1 compares the hydrogen recovery for a prior art process and the process of this invention for a plant producing 1000 kilotonnes of ethylene per year. It also compares the total compressor energy requirement for the two processes (measured as the sum of the cracked gas compressor and the ethylene and propylene refrigeration compressor horsepower requirements). The process of this invention recovers approximately 2,200 lb/hr more hydrogen product than the prior art process, with a relatively modest 275 HP increase in energy requirement. The value of the additional hydrogen product more than offsets the slightly higher energy use of the process of this invention.

TABLE 1
Hydrogen Production, Hydrogen Recovery, and Energy Requirement

| | Prior Art Design | |
|---|---|---|
| | (Manley U.S. Pat. No. 5,675,054) | This Invention |
| Hydrogen Product | 9939 | 12186 |
| flow (lb/hr) | | |
| Hydrogen Recovery | 60.2% | 73.8% |
| to Product (%) | | |
| Total Compressor | 99736 | 100011 |
| Horsepower (HP) | | |

Conditions and compositions of streams shown in FIG. 2 are given in Table 2 and exchanger duties are given in Table 3. The data in Table 2 shows that the arrangement in FIG. 2 produces a partial separation of the hydrogen and methane that are present in the ethylene distributor overhead.

TABLE 2
Flows and Conditions for Streams in FIG. 2

| | Stream No. | | | | | | | | | |
|---|---|---|---|---|---|---|---|---|---|---|
| | 201 | 202 | 203 | 204 | 205 | 206 | 208 | 210 | 211 | 213 |
| Temperature (Deg F.) | −12.7 | −73.4 | 36.0 | −11.4 | −145.0 | −145.0 | −145.0 | −206.2 | −149.9 | −142.1 |
| Pressure (psig) | 515 | 500 | 515 | 511 | 498 | 498 | 498 | 495 | 498 | 480 |
| Molar flows (lb mol/hr) | | | | | | | | | | |
| CO | 34.4 | 32.6 | 0.0 | 1.8 | 3.7 | 2.2 | 29.0 | 28.5 | 0.5 | 4.2 |
| HYDROGEN | 8458.2 | 8254.6 | 0.0 | 203.6 | 147.3 | 88.4 | 8107.3 | 8087.3 | 20.0 | 167.3 |
| METHANE | 5349.3 | 4713.9 | 0.9 | 634.5 | 1643.5 | 986.1 | 3070.5 | 2829.1 | 241.4 | 1883.8 |
| ETHYLENE | 16353.0 | 5178.1 | 4423.3 | 6751.6 | 4565.9 | 2739.5 | 612.2 | 11.0 | 601.2 | 2.1 |
| ETHANE | 5956.9 | 1.6 | 2533.8 | 3421.6 | 1.5 | 0.9 | 0.1 | 0.0 | 0.1 | 0.0 |
| ACETYLENE | 0.0 | 0.0 | 0.0 | 0.0 | 0.0 | 0.0 | 0.0 | 0.0 | 0.0 | 0.0 |
| PROPYLENE | 2.9 | 0.0 | 1.4 | 1.6 | 0.0 | 0.0 | 0.0 | 0.0 | 0.0 | 0.0 |
| PROPANE | 0.0 | 0.0 | 0.0 | 0.0 | 0.0 | 0.0 | 0.0 | 0.0 | 0.0 | 0.0 |
| PROPDIENE | 0.0 | 0.0 | 0.0 | 0.0 | 0.0 | 0.0 | 0.0 | 0.0 | 0.0 | 0.0 |
| METHYLACETYLENE | 0.0 | 0.0 | 0.0 | 0.0 | 0.0 | 0.0 | 0.0 | 0.0 | 0.0 | 0.0 |
| | Stream No. | | | | | | | | | |
| | 214 | 215 | 216 | 217 | 218 | 219 | 220 | 221 | 222 | 223 |
| Temperature (Deg F.) | 18.1 | −206.2 | −206.2 | −219.7 | −219.7 | −161.3 | −161.3 | −212.0 | −213.3 | −161.3 |
| Pressure (psig) | 485 | 495 | 495 | 490 | 42 | 487 | 41 | 135 | 42 | 41 |
| Molar flows (lb mol/hr) | | | | | | | | | | |
| CO | 0.0 | 4.4 | 24.1 | 16.0 | 8.1 | 16.0 | 8.1 | 8.5 | 8.5 | 8.5 |
| HYDROGEN | 0.0 | 1236.4 | 6850.9 | 6093.0 | 757.9 | 6093.0 | 757.9 | 1403.7 | 1403.7 | 1403.7 |
| METHANE | 1.0 | 432.5 | $$ | 255.2 | 2141.4 | 255.2 | 2141.4 | 2316.3 | 2316.3 | 2316.3 |
| ETHYLENE | 5165.0 | 1.7 | 9.3 | 0.0 | 9.3 | 0.0 | 9.3 | 3.7 | 3.7 | 3.7 |
| ETHANE | 1.6 | 0.0 | 0.0 | 0.0 | 0.0 | 0.0 | 0.0 | 0.0 | 0.0 | 0.0 |
| ACETYLENE | 0.0 | 0.0 | 0.0 | 0.0 | 0.0 | 0.0 | 0.0 | 0.0 | 0.0 | 0.0 |
| PROPYLENE | 0.0 | 0.0 | 0.0 | 0.0 | 0.0 | 0.0 | 0.0 | 0.0 | 0.0 | 0.0 |
| PROPANE | 0.0 | 0.0 | 0.0 | 0.0 | 0.0 | 0.0 | 0.0 | 0.0 | 0.0 | 0.0 |
| PROPDIENE | 0.0 | 0.0 | 0.0 | 0.0 | 0.0 | 0.0 | 0.0 | 0.0 | 0.0 | 0.0 |
| METHYLACETYLENE | 0.0 | 0.0 | 0.0 | 0.0 | 0.0 | 0.0 | 0.0 | 0.0 | 0.0 | 0.0 |

TABLE 3
Heat Exchanger Duties for FIG. 2

| Exchanger | Service | Duty (mBTU/hr) |
|---|---|---|
| E201 | Ethylene Distributor Condenser | −82.71 |
| E202 | Ethylene Distributor Reboiler | 19.61 |
| E203 | Cracked Gas Chiller | −36.03 |
| E204 | Demethanizer Condenser | −5.69 |
| E205 | Demethanizer Reboiler | 17.67 |
| E206 | First Stage Hydrogen Recovery | −5.82 |
| E207 | Second Stage Hydrogen Recovery | −4.02 |
| C203 | Dephlegmator | −8.68 |

Table 4 compares the hydrogen and methane contents of stream 202 (net ethylene distributor overhead), stream 219 (the final hydrogen product stream) and the combination of fuel streams (streams 220 and 223). This table demonstrates that through the application of this invention, about 74% of the hydrogen present in stream 202 is recovered as salable hydrogen product, while only about 26% of the hydrogen present in stream 202 is lost into the fuel streams. The drawings contain depictions of certain embodiments of this invention. All major separation, heating, and cooling steps have been shown.

TABLE 4
Hydrogen and Methane Recovery

| | | Stream and Stream Number | | | | |
|---|---|---|---|---|---|---|
| | | Ethylene | H2 | Fuel | Fuel | Total Fuel |
| | | Distr. Ovhd | Product | Stream 1 | Stream 2 | Stream |
| | | 202 | 219 | 220 | 223 | (220 + 223) |
| Hydrogen Flow | lbmol/hr | 8254.6 | 6093.0 | 757.9 | 1403.7 | 2161.6 |
| Methane Flow | lbmol/hr | 4713.9 | 255.2 | 2141.4 | 2316.3 | 4457.7 |
| Hydrogen Recovery | % | | 73.8% | | | 26.2% |
| Methane Recovery | % | | 5.4% | | | 94.6% |

1. A process for recovering hydrogen comprising the steps of:
a. Introducing a mixed hydrocarbon feed stream comprising a mixture of hydrogen, methane, and C2 components into an ethylene distributor column to obtain a first stream;
b. Subjecting at least a portion of said first stream to a separation technique to produce a second stream substantially hydrogen enriched and substantially methane depleted and a third stream substantially hydrogen depleted and substantially methane enriched;
c. Recovering a hydrogen product from said second stream.

2. The process of claim 1 wherein said first stream enters at least one partial condenser to provide at least a portion of the reflux to said ethylene distributor column prior to step (b).

3. The process of claim 1 wherein said third stream is introduced into a second separation column to produce a fourth stream substantially methane enriched and a fifth stream substantially ethylene enriched.

4. The process of claim 1 wherein said first stream comprises a mixture of hydrogen, C1, and ethylene and is substantially free of ethane.

5. The process of claim 3 wherein said second separation column is a demethanizer.

6. The process of claim 1 wherein said separation step comprises rectification, condensation, membrane separation, dephlegmation, adsorption, either individually or in combinations thereof.

7. The process of claim 3 wherein said fourth stream comprises essentially little or no ethylene.

8. A process for recovering hydrogen and providing refrigeration duty comprising the steps of:
a. Introducing a mixed hydrocarbon feed stream comprising a mixture of hydrogen, methane, and C2 components into an ethylene distributor to obtain a first stream;
b. Subjecting at least a portion of said first stream to a separation technique to produce a second stream substantially hydrogen enriched and substantially methane depleted and a third stream substantially hydrogen depleted and substantially methane enriched;
c. Introducing said third stream into a second separation column to recover a fourth stream substantially methane enriched;
d. Directing said fourth stream to an expansion device to produce a lower-temperature, lower pressure fifth stream
e. Reheating said lower-temperature, lower-pressure fifth stream to provide cooling duty to the overall process.
f. Recovering a hydrogen product from said second stream.

9. The process of claim 8 wherein said first stream enters at least one partial condenser to provide at least a portion of the reflux to said ethylene distributor column prior to step (b).

10. The process of claim 8 wherein said first stream comprises a mixture of hydrogen, C1, and ethylene and is substantially free of ethane.

11. The process of claim 8 wherein said second separation column is a demethanizer.

12. The process of claim 8 wherein said separation step comprises rectification, condensation, membrane separation, dephlegmation, adsorption, either individually or in combinations thereof.

13. The process of claim 8 wherein said fourth stream comprises essentially little or no ethylene.

14. The process of claim 8 wherein a portion of said second stream of step (b) may be directed to said expansion device of step (d).
